(19) United States
(12) Patent Application Publication
(10) Pub. No.: US 2025/0314726 A1
(43) Pub. Date: Oct. 9, 2025

(54) Magnetic Resonance Relaxometry (MRR) Measurement to Predict Chondrogenic Potential of Mesenchymal Stem Cells (MSCS)

(71) Applicants: Massachusetts Institute of Technology, Cambridge, MA (US); National University of Singapore, Singapore (SG)

(72) Inventors: Jongyoon Han, Bedford, MA (US); Zheng Yang, Singapore (SG); Dahou Yang, Singapore (SG); Yingnan Wu, Singapore (SG); Eng Lee, Singapore (SG)

(21) Appl. No.: 18/865,877

(22) PCT Filed: May 18, 2023
(86) PCT No.: PCT/US2023/067169

§ 371 Date: Jun. 30, 2025

Related U.S. Application Data
(60) Provisional application No. 63/343,329, filed on May 18, 2022.

(51) Int. Cl. G01R 33/44; C12N 5/077; G01N 24/08; G01R 33/30; G01R 33/465

(52) U.S. Cl. CPC G01R 33/448; C12N 5/0655; G01N 24/085; G01R 33/302; G01R 33/465; C12N 2506/1353

(57) ABSTRACT

Chondrogenic potential of mesenchymal stem cells (MSCs) can be determined using magnetic resonance relaxometry by measuring T2 relaxation value of the MSCs.

### CLAIM OF PRIORITY

The application claims priority to U.S. Provisional Patent Application No. 63/343,329, filed May 18, 2022, which is incorporated by reference in its entirety.

### FIELD OF THE INVENTION

The invention features systems and methods for non-invasive cell analysis.

### BACKGROUND

There is wide donor-to-donor variability affected by donor's age and disease status, as well as tissue source variation, in MSC functionality and differentiation capacity (see, for example, Zha et al 2021), which could be further compounded by the influence of in vitro culture conditions. This heterogeneity poses a significant obstacle in the research and application of MSCs to yield consistent and effective clinical outcomes. To date, apart from subjecting MSCs to the lengthy and laborious chondrogenic differentiation, there are currently no critical quality attributes (CQAs) that can rapidly predict the cartilage forming ability of donor-derived and expanded MSCs.

### SUMMARY

In general, magnetic resonance relaxometry (MRR) measurement can be used to predict or identify chondrogenic potential of mesenchymal stem cells (MSCs). For example, measurement of properties by MRR, particularly T₂, can be used to identify MSCs with superior chondrogenesis, or cartilage regeneration capability.

In one aspect, a method of identifying chondrogenic potential of mesenchymal stem cells can include loading a liquid sample including a plurality of mesenchymal stem cells in a sensor, placing the sensor including the liquid sample within or nearby a detection coil of a magnetic resonance relaxometry device, determining a T₂ value for the liquid sample, and identifying mesenchymal stem cells with high chondrogenic potential when the T₂ value is greater than or equal to a first threshold value and mesenchymal stem cells with low chondrogenic potential when the T₂ value is less than or equal to a second threshold value.

In another aspect, a system for identifying chondrogenic potential of mesenchymal stem cells can include a magnetic resonance relaxometry device configured to determining a T₂ value for a liquid sample including a plurality of mesenchymal stem cells in a sensor and identifying mesenchymal stem cells with high chondrogenic potential when the T₂ value is greater than or equal to a first threshold value and mesenchymal stem cells with low chondrogenic potential when the T₂ value is less than or equal to a second threshold value.

In certain circumstances, the sensor can be a tube or chamber.

In certain circumstances, the first threshold value can be greater than the second threshold value.

In certain circumstances, a gap between the first threshold value and the second threshold value can be 10 ms, 20 ms, 30 ms, 40 ms, 50 ms, 60 ms, 70 ms, 80 ms, 90 ms or 100 ms.

In certain circumstances, the first threshold value can be 1200 ms.

In certain circumstances, the second threshold value can be 1100 ms.

In certain circumstances, the T₂ value can be correlated with the formation of a cartilaginous matrix component. In certain circumstances, the cartilaginous matrix component includes sulphated glycoaminoglycan (sGAG), type II collagen (COL2), or mixtures thereof.

In certain circumstances, the T₂ value can be correlated with quantitative production of sGAG.

In certain circumstances, the T₂ value can be correlated with quantitative production of COL2.

In certain circumstances, the magnetic resonance relaxometry device can include a radio frequency probe.

In certain circumstances, determining the T₂ value can include supplying a train of pulses over a period of less than one minute.

In certain circumstances, determining the T₂ value can include obtaining and averaging 2 to 70 scans, 4 to 60 scans, or 6 to 40 scans. For example, determining the T₂ value can include obtaining and averaging 10 to 30 scans.

In certain circumstances, the method can include separating mesenchymal stem cells with high chondrogenic potential from mesenchymal stem cells with low chondrogenic potential.

In certain circumstances, the mesenchymal stromal cells include bone marrow derived mesenchymal stem cells.

In certain circumstances, a detection region of the magnetic resonance relaxometry device can include a volume of less than about 1 μL of the sample. For example, the volume can be less than 0.1 μL, less than about 0.01 μL, less than about 0.001 μL, or less than about 0.0001μL, In certain circumstances, the volume can be about 1 pL to 10 pL.

In certain circumstances, the liquid sample can be free of paramagnetic or ferromagnetic materials, including paramagnetic or ferromagnetic metal ions or compounds thereof.

In certain circumstances, the system can include a cell production device as a source for the plurality of mesenchymal stem cells.

In certain circumstances, the cell production device can include a cell culture vessel, for example, a plate, a well, or a chamber.

In certain circumstances, the cell production device can include an incubator.

In certain circumstances, the system can include a cell separation device, for example, a microfluidic device or a cell sorter.

In certain circumstances, the cell production device can be a source for the plurality of mesenchymal stem cells. For example, the cell production device can include a cell culture system.

In certain circumstances, the liquid sample can be contained in a microcapillary.

Other aspects, embodiments, and features will be apparent from the following description, the drawings, and the claims.

### BRIEF DESCRIPTION OF DRAWINGS

FIG. 1A depicts a schematic of a Magnetic Resonance Relaxometry (MRR) device.

FIG. 1B depicts a sample for Magnetic Resonance Relaxometry (MRR) and exemplary T₂ values for different cell types.

FIG. 2 depicts a schematic of system including a Magnetic Resonance Relaxometry (MRR) device and a cell production device.

FIGS. 3A-3B show a CPMG (Carr-Purcell-Meiboom-Gill) pulse sequence for measuring the T₂ values.

FIG. 4 is a graph showing the MRR measurement of six MSC donors, taken just before subjection to chondrogenic differentiation.

FIG. 5A depicts photographs of formation of sGAG and Col 2 as indicated by Safranin O staining, and Col 2 immuno-staining. Images shown are representative of n=3 per donor.

FIG. 5B depicts graphs showing quantification of cartilaginous extracellular matrix macromolecules, sGAG and COL2 generated by MSC after 21 days of differentiation. Data shown represent means±SD, n=3 per donors.

FIGS. 6A-6B are graphs depicting R² coefficient of determination of MRR T₂ levels and differentiated MSC quantitative production sGAG or type II collagen, respectively.

### DETAILED DESCRIPTION

Mesenchymal stem cells (MSCs) offer an attractive cell source for cartilage tissue engineering due to their relative ease of derivation, proliferative capacity and differentiation potential. However, there is wide donor-to donor variability in MSC functionality and differentiation capacity, which could have accounted for the inconsistent clinical outcomes (see, for example, Zha et al 2021, which is incorporated in its entirety). To date, apart from subjecting MSCs to the lengthy and laborious chondrogenic differentiation, there are no other critical quality attributes (CQAs) that can rapidly predict the cartilage forming ability of donor-derived and expanded MSCs. The systems and methods described herein include the use of a benchtop magnetic resonance relaxometry (MRR) system (see, for example, Peng et al 2014, which is incorporated in its entirety, to measure the proton magnetic resonance relaxation of MSCs as a means for rapid and labelled-free prediction on the chondrogenic efficacy MSCs. Culture expanded MSCs were subjected to MRR measurement using a portable bench-top MRR system prior to being subjected to three weeks differentiation culture. The chondrogenic potential of the expanded MSCs was determined by the formation of cartilaginous matrix components, sulphated glycoaminoglycan (sGAG) and type II collagen (COL2). MRR T₂ levels was then correlated to the quantitated matrix levels. A strong positive correlation between MRR T2 levels of the undifferentiated MSC and differentiated MSC quantitative production of sGAG and COL2 was found, with R² coefficient of determination at 0.95 and 0.96, respectively. This result indicate that T₂ value has very high correlation to the chondrogenic capability of MSCs. Rapid MRR T₂ measurement could serve as an efficacy CQA and PAT for identifying MSCs with superior cartilage regeneration capability.

The results described herein indicate that T₂ value greater than 1200 ms predict MSC of high chondrogenic capacity; while T₂ value less than 1100 ms predict MSC of poor chondrogenic potential.

MRR measurement is rapid (<1 min/test), requires minimum manipulation (does not require any chemical or immunolabeling), is sensitive and highly reproducible. This has tremendous advantage compared to conventional determination of MSC chondrogenic ability by either mRNA analysis (takes days) or qualitative or quantitative matrix analysis (takes 2-3 weeks), which is laborious and time-consuming. It allows rapid identification of quality MSC with superior cartilage regeneration potential. In addition, it can be used as monitoring CQA and PAT for further development of media and adaptive culture of MSCs specifically for cartilage regeneration application. The discoveries described herein advance over current existing methods, including:

(1) The selection of functional MSC subpopulations for the treatment of cartilage damage targeting surface markers such as CD271, CD146, CD105, and Stro-1. Surface expression of these markers, alone or in various combination, has been identified to represent MSC subpopulation with superior clonogenic potential (see, for example, Joes et al 2002; Chang et al 2013; Mifune et al 2013; Mabuchi et al 2013; and Li et al 2019, each of which is incorporated in its entirety). Fluorescence-activated cell sorting (FACS) was used to isolate more functional MSC subpopulations from the heterogenous pool of donor's derived MSCs that exhibited robust multilineage differentiation and self-renewal potency. FACS selection of MSCs is however manipulative, requires immunolabelling of cells, and targets the selection of MSC subpopulation within the heterogenous pool of MSCs from a single donor. Analysis of surface marker expression does not provide information on donor-to-donor variability in MSC potency, let alone the chondrogenic cacpability. In addition, the expression of these markers tends to gradually decrease with passaging in vitro, rendering them as obsolete attributes for expanded MSCs; and

(2) GSTT1, genomic biomarker that identifies human bone marrow-derived MSCs with high scalability. A loss of the gene encoding glutathione S-transferase theta 1 (GSTT1) has been identified in hMSC donors with high-growth-capacity (see, for example, Sathiyanathan et al 2020, which is incorporated in its entirety). These GSTTI-null hMSCs demonstrated increased proliferative rates, clonogenic potential, and longer telomeres compared with low-growth capacity hMSCs that were GSTT1-positive. However, the lack of GSTT1 genes does not confer significant advantage in the multidifferentiation ability of MSCs. Thus, GSTT1 identifies MSC with high scalability, but does not reveal the multipotent, let alone the chondrogenic cacpability of the MSCs.

In summary, the systems and methods described herein lead to one or more of the following advantageous and surprising results:
(1) Direct correlation of undifferentiated MSC MRR transverse relaxation time (T₂) with differentiated MSC quantitative production of type II collagen and sGAG;
(2) The use of a benchtop magnetic resonance relaxometry (MRR) system to measure the proton magnetic resonance relaxation of mesenchymal stem cells (MSCs) as a mean to predict the chondrogenic efficacy MSCs; and
(3) The MRR T₂ chondrogenic prediction are not limited to bone marrow derived MSCs, but MSCs derived from other tissue sources, and include MSCs subjected to various manipulations and treatments.

The rapid and label-free measurement of the proton magnetic resonance relaxation of MSCs by the benchtop magnetic resonance relaxometry (MRR) system can be deployed as a mean to predict the chondrogenic efficacy MSCs. This could serve as:
(i) functional CQA and PAT for the identification of “optimum” MSCs with superior cartilage regeneration potential; and
(ii) monitoring CQA and PAT for further development of media and adaptive culture of MSCs specifically for cartilage regeneration applications.

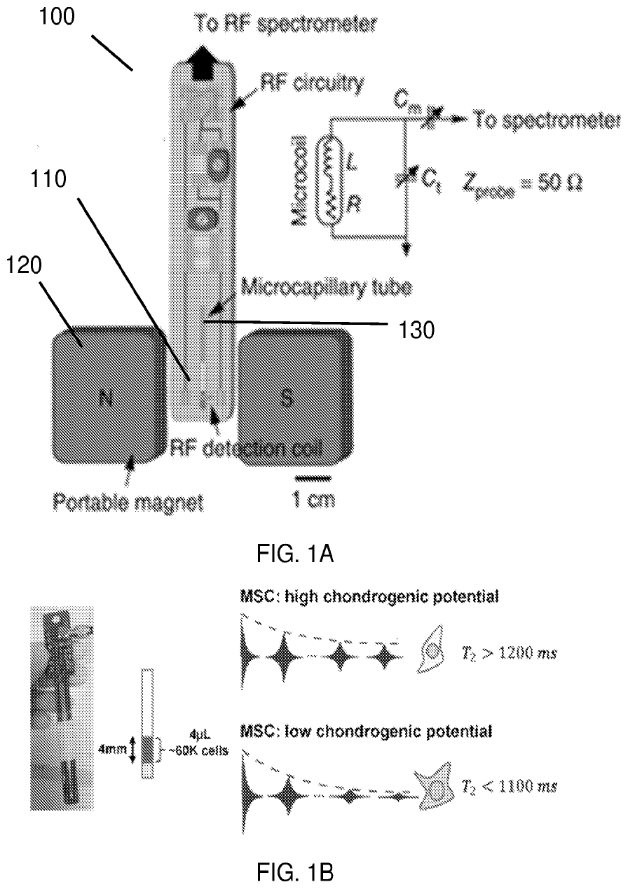

A device for performing magnetic resonance relaxometry is described, for example, in U.S. Pat. No. 10,429,467, which is incorporated by reference in its entirety. Referring to FIGS. 1A and 1B, a device can include an MRR system. FIG. 1A is a schematic of a Magnetic Resonance Relaxometry (MRR) system 100 in accordance with one aspect of this disclosure. The system 100 can include a Field-Programmable Gate Array-based (FPGA-based) radio frequency (rf) spectrometer to control the MRR system 100, a first direct digital synthesis module for generation of radio frequency pulses, a transmitter (TRANS) for transmission of the generated radio frequency pulses to a radio frequency (rf) probe and detection coil 110, a receiver (RCVR) for receiving resonance information from the radio frequency probe, a first power amplifier (PA), a pre-amplifier (p-amp), a duplexer (Dup) for transmitting a high power excitation pulse to the rf probe in the transmission mode and for isolating the high power excitation pulse from the receiver during receiving mode, and a magnet system 120. A sample 130 can be placed in a sensor, surch as a tube or chamber, for example, a microcapillary tube, that can be positioned in an RF detection coil. In many embodiments, the FPGA-based rf spectrometer can include a pulse programmer (PPG) adapted to control the FPGA-based rf spectrometer and a second direct digital synthesis (DDS). The second DDS can generate a fixed intermediate frequency (IF). The first DDS can be configured to generate a variable desired frequency. In accordance with one aspect of this disclosure, the FPGA-based spectrometer may use the design set forth in Takeda K. (2007), “A highly integrated FPGA-based nuclear magnetic resonance spectrometer,” Rev Sci Instrum 78 (3): 033103; and/or in Takeda K. (2008) “OPENCORE NMR: open-source core modules for implementing an integrated FPGA-based NMR spectrometer,” Journal of Magnetic Resonance 192 (2): 218-229, the teachings of which two references are incorporated by reference in their entirety.

In order to facilitate processing of information to and from the MRR system 100, the FPGA-based rf spectrometer is couplable to at least one external electronic device which may, for example, include a personal computer, mobile phone and/or a portable electronic tablet. Coupling between the MRR system 100 and the at least one external electronic device may be by way of at least one of USB, HDMI and/or wireless connection means such as Wi-Fi and/or Bluetooth.

In conventional NMR systems, the major cost of instrumentation lies on the superconducting magnet (or permanent magnet) and rf-spectrometer. In accordance with one aspect of this disclosure, the whole system may cost less than $2500; in which the majority of the cost lies on the FPGA chip ($1000 each), external GHz-clock ($250 each), DDS (Analog-Device; AD9858, $400 each), 1-Watt power amplifier ($100), pre-amplifier ($50), RCVR (AD8343, $4 each), TRANS (AD834, $20 each, and AD8343) and USB (FT2232D, $10 each). Indicated in the parentheses is the cost of the main electronic component used. Others periphery components such as pin connectors (e.g., SMA), capacitors, rf-switches, rf-transformers and rf-filters cost less than $10 each.

The MRR system 100 may be adaptable to operate in various modes to detect NMR-active nuclei such as proton, fluorine, phosphorus and carbon. The magnetic field used in each mode in which the MRR system 100 operates depends on which nuclei are to be detected. Depending on the mode of operation, the MRR system 100 can operate at a magnetic field of between approximately 0.1 and 3 Tesla (T) which can correspond to between approximately 1 and 150 MHz. For instance, when the MRR system 100 is operating in a proton NMR mode, the magnetic field is approximately 0.76 T which corresponds to approximately 31.9 MHz for proton NMR frequency.

The MRR system 100 can be controlled by the FPGA-based rf spectrometer which comprises the pulse programmer and the second DDS. As compared to CMOS technology, FPGA provides the advantages of re-programmability. The FPGA-based rf spectrometer may, for example, be programmable using tools and software provided by vendors such as Altera Corporation of San Jose, Calif., U.S.A. and Xilinx, Inc. of San Jose, Calif., U.S.A. In an exemplary embodiment, the FPGA chip can include the EP3C80F780C8N, Cyclone III (Altera) embedded on a breadboard (ACM-202-80C8, HumanData, Japan). This chip has 81000 logic elements and is capable of producing 3 independent if-outputs, when fully utilized.

The pulse programmer can generate high power excitation rf pulses. The generated rf pulses then pass through the first power amplifier to produce optimized rf-power for a duration of approximately between 1 and 1000 microseconds to excite all the nuclei effectively. The high power rf pulses are transmitted to the rf probe and will be discussed further herein.

In an exemplary operation, power used for liquid state and solid-state NMR is approximately between 0.1 W and 10 W and approximately between 100 W and 1000 W, respectively. A “strong” power amplifier is often indispensable in MRR systems and such “strong” power amplifiers are often bulky, and require high power consumption, thereby posing serious limitation for field work. For example, a novel and lightweight 1-Watt power amplifier can be constructed on a 4 cm by 4 cm printed circuit board. A solenoid type microcoil (inner diameter 700 to 1000 μm, for example, 750, 800, 850, 900 or 950 μm) can be further employed to generate a strong oscillating magnetic field, B₁, and picks up a signal from the free induction decay (FID) or spin-echo. By employing the duplexer, the high power excitation if pulses that are to be transmitted to the rf-probe in the transmission mode can be isolated from the receiver or detection coil 110 during the receiving mode. The FID/spin-echo is then amplified by a pre-amplifier (AMP-75+, Mini Circuits, USA) with a gain of 20 dB and noise figure of 2.83, and finally filtered by appropriate low pass filter before going into the receiver circuit. FID is the observable NMR signal generated by non-equilibrium nuclear spin magnetization precessing about the static magnetic field (conventionally along z-axis). This non-equilibrium magnetisation can be induced, by applying a pulse of resonant radio-frequency close to the Larmor frequency of the nuclear spins. Spin-echo is the refocusing pulse after a single 90-degree inversion followed by inverting them by an 180-degree pulse at resonant.

The magnet system 120 may be portable and light weight (for example, about 60 g) and adaptable to produce a high static field. The magnet system 120 may comprise at least one magnet disposed adjacent to the rf probe. Alternative embodiments include having at least two magnets disposed adjacent to the rf probe. The rf probe can be disposed between the at least two magnets. The magnet system 120 can comprise a permanent magnet and/or an electromagnet. Permanent magnets used in the magnet system 120 may, for example, include Neodymium based magnets.

A detection region of the magnetic resonance relaxometry device can include a volume of less than about 1 μl of the sample for detection. For example, the sample can be provided in sensor, such as a capillary tube or microcapillary tube or a chamber. The sample can be sealed from the ambient environment, reducing exposure to oxygen and other materials that could negatively impact the ability of the magnetic resonance relaxometry device to detect senescent cells. The sample can include a buffer solution that is free of any paramagnetic iron compounds, which can be important in order to give an accurate result.

As described herein, and building on the description of the device described above, a method of detecting senescent cells can include loading a liquid sample including a plurality of cells in a sensor, placing the sensor including the liquid sample within a detection coil of a magnetic resonance relaxometry device, and determining a T₂ value to detect an amount of senescent cells in the liquid sample. Details of determining a T₂ value to detect an amount of senescent cells are described below. For example, the T₂ value can decrease as a number of senescent cells increases. In certain circumstances, determining a T₂ can include measuring a magnetic susceptibility index of the cells.

The current μMRR set up is only suitable for measurement of cell suspension, for example, in a liquid sample. A μMRR set up can be designed to adherent cells on cell culture plates. The devices, systems and methods described herein can be used to develop and improve cell therapies by facilitating removal of senescent cells from cell populations through simplifying the identification process for senescent cells and using that approach in conjunction with cell manipulation technologies.

One way of determining a T₂ value to identify mesenchymal stem cells with high chondrogenic potential and mesenchymal stem cells with low chondrogenic potential. In certain circumstances, determining a T₂ can be correlated with quantitative production of sGAG, quantitative production of COL2, or both. The magnetic resonance relaxometry device can include a radio frequency probe that can be configured identifying mesenchymal stem cells with high chondrogenic potential when the T₂ value is greater than or equal to a first threshold value and mesenchymal stem cells with low chondrogenic potential when the T₂ value is less than or equal to a second threshold value.

A number of approaches can be taken to improve the accuracy of the T₂ value. For example, determining the T₂ value can include obtaining and averaging a plurality of scans. Up to 100 (or more) scans can be averaged. The number of scans can be less than 80, less than 70, or less than 60. More typically, 10 to 50 scans can be averaged. As a minimum, under certain circumstances, 2 scans, 4 scans, 5 scans, 8 scans, 10 scans, 12 scans, 14 scans, 16 scans, 18 scans, 20 scans, 22 scans, 24 scans, 26 scans, 28 scans, or 30 scans can be averaged. In certain circumstances, the T₁ relaxation time can be used in conjunction with T₂ value to identifying mesenchymal stem cells with high chondrogenic potential when the T₂ value is greater than or equal to a first threshold value and mesenchymal stem cells with low chondrogenic potential when the T₂ value is less than or equal to a second threshold value.

The devices, systems and methods described herein can be used to identifying mesenchymal stem cells with high chondrogenic potential when the T₂ value is greater than or equal to a first threshold value and mesenchymal stem cells with low chondrogenic potential when the T₂ value is less than or equal to a second threshold value. The first threshold value is greater than the second threshold value. In general, mesenchymal stem cells with high chondrogenic potential have a T₂ value greater than the T₂ value of the mesenchymal stem cells with low chondrogenic potential. In certain circumstances using the methods described herein, a cut off T₂ value can be 1500 ms. In order to more clearly distinguish mesenchymal stem cells with high chondrogenic potential from mesenchymal stem cells with low chondrogenic potential, a gap can be used between the two T₂ values. The gap can be 10 ms, 20 ms, 30 ms, 40 ms, 50 ms, 60 ms, 70 ms, 80 ms, 90 ms or 100 ms. The gap can have a variance of up to 10%, 8%, 5%, or 2%. For example, the first threshold value can be 1200 ms. The second threshold value can be 1100 ms. Each threshold value can vary by up to 8%, 5%, or 2%. T₂ value can be correlated with the formation of a cartilaginous matrix component. In certain circumstances, the cartilaginous matrix component includes sulphated glycoaminoglycan (sGAG), type II collagen (COL2), or mixtures thereof.

The systems and methods described herein can be used to develop an assay. The assay could be used as a release assay at the end of MSC production, before implantation. Alternatively, the assay can be done in the middle of the cell production, monitoring the progress of the culture and identify any deviation from the range of acceptable values, to identify the bad batches early. The assay can be used in conjunction with a cell production device can be a source for the plurality of mesenchymal stem cells. For example, the cell production device can include a cell culture system.

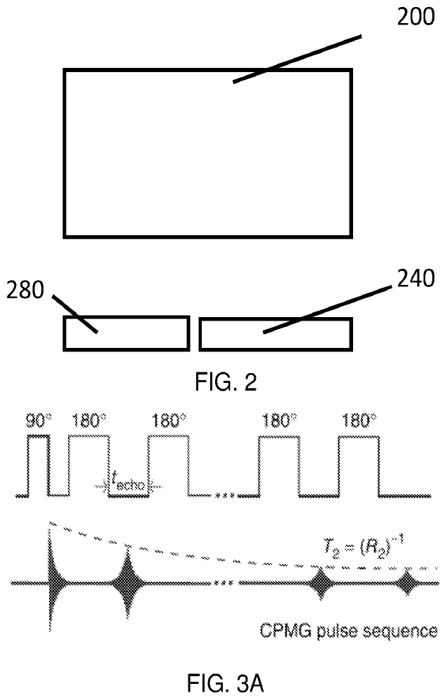

Referring to FIG. 2, a system described herein can include magnetic resonance relaxometry device 200 and cell production device 240. For example, a system for identifying chondrogenic potential of mesenchymal stem cells can include a magnetic resonance relaxometry device configured to determine a T₂ value for the liquid sample, and identify mesenchymal stem cells with high chondrogenic potential when the T₂ value is greater than or equal to a first threshold value and mesenchymal stem cells with low chondrogenic potential when the T₂ value is less than or equal to a second threshold value. The cell production device can include a cell culture vessel. The cell culture vessel can include a plate, a well, or a chamber which can be used to culture and grow the mesenchymal stem cells described herein.

Referring to FIG. 2, the system can include a cell separation device 280. The cell separation device can be used in the method described herein for separating mesenchymal stem cells with high chondrogenic potential from mesenchymal stem cells with low chondrogenic potential. The cell separation device can include a microfluidic device or a cell sorter that can be used to implement the separating based on identification of the mesenchymal stem cells with high chondrogenic potential using the methods described herein.

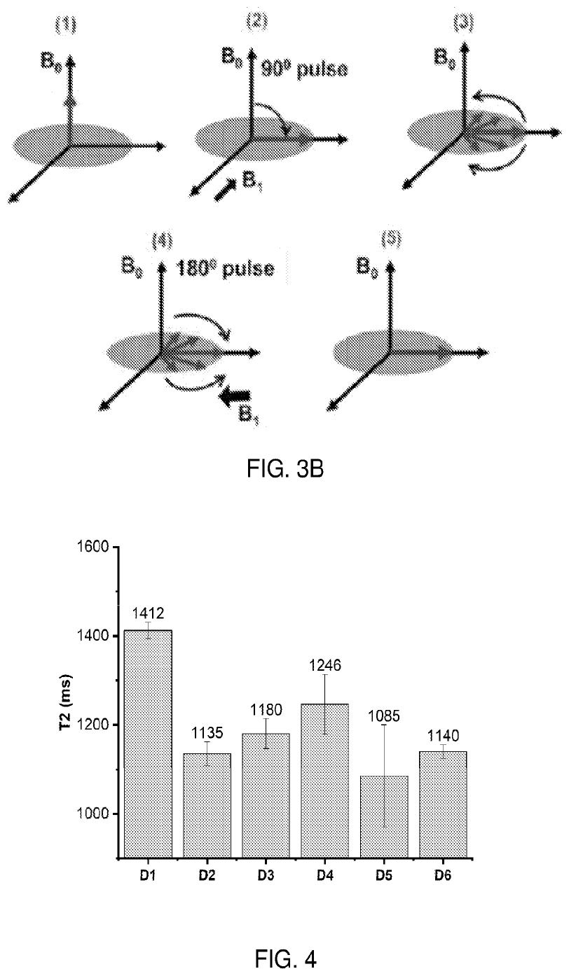

Referring to FIGS. 3A and 3B, the CPMG (Carr-Purcell-Meiboom-Gill) pulse sequence for measuring the T₂ values can work efficiently in inhomogeneous magnetic fields produced by permanent magnet of MRR. A train of radiofrequency pulses are applied to the proton nuclei at the resonance frequency of 21.65 MHz with the inter echo time interval t echo and it is repeated for thousands of echoes until relaxes over time. This decayed height of echoes over time is called transverse relaxation time T₂.

FIGS. 1A-1B show non-invasive and rapid magnetic resonance relaxometry (MRR) measurement to predict chondrogenic potential of MSC. FIG. 1A is a schematic showing a MRR system consists of a permanent magnet that provides a strong magnetic field (B₀). A home-built radiofrequency (RF) detection probe is connected to an RF spectrometer. The MSC sample in the microcapillary tube is placed in the RF detection coil for T₂ measurements. FIG. 1B shows a microcapillary tube contains the 4 μl of MSC sample in the 4 mm detection range of the RF detection coil. The typical cell number required is 60,000 MSCs within the detection volume. The bottom grey color is the crystoseal to seal the microcapillary tube. The right side shows the 1H spin-spin relaxation time T₂ of high chondrogenic potential MSCs, and low chondrogenic potential MSC with decreased T₂.

FIG. 2 depicts a schematic of system including a Magnetic Resonance Relaxometry (MRR) device and a cell production device as a source of plurality of mesenchymal stem cells.

FIGS. 3A and 3B show a CPMG (Carr-Purcell-Meiboom-Gill) pulse sequence for measuring the T₂ values, which works efficiently in inhomogeneous magnetic fields produced by the permanent magnet of μMRR. A train of radiofrequency pulses is applied to the proton nuclei at the resonance frequency of 21.65 MHz with the inter echo time interval techo. It is repeated for thousands of echoes until an equilibrium is reached. This decaying peak height of successive echoes over time is called transverse relaxation time T₂.

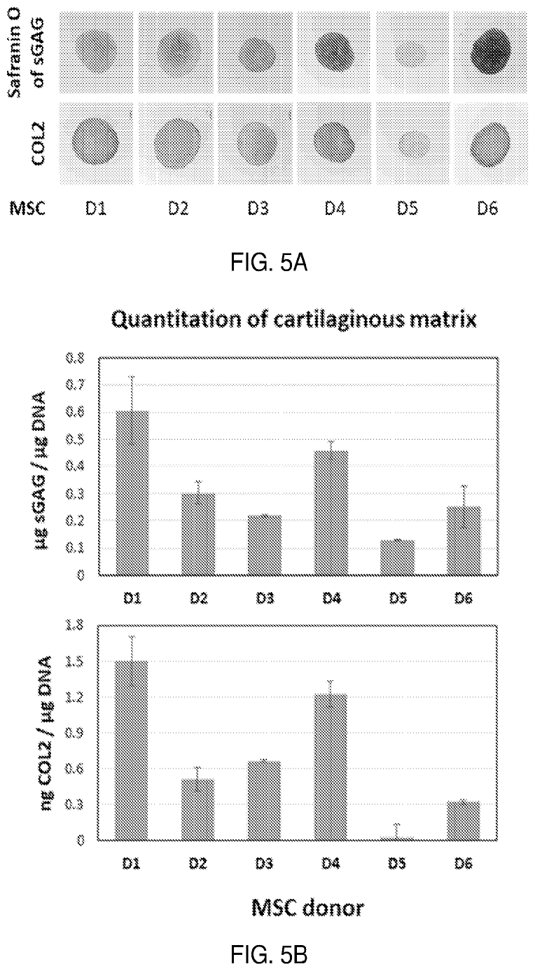

FIG. 4 shows a graph of MRR measurement of six MSC donors at passage 4, taken just before subjection to chondrogenic differentiation.

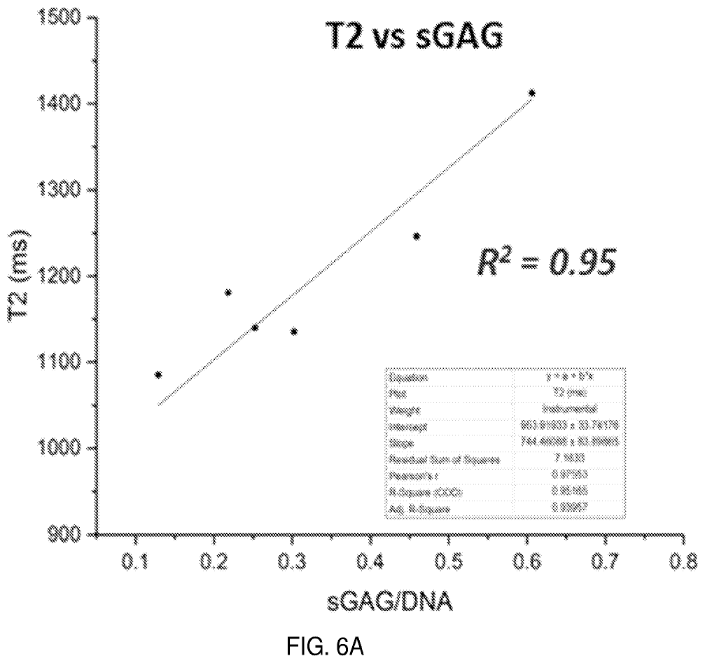

FIG. 5A shows formation of sGAG and Col 2 as indicated by Safranin O staining, and COL2 immuno-staining. Images shown are representative of n=3 per donor. FIG. 5B shows quantification of cartilaginous extracellular matrix macromolecules, sGAG and COL2 generated by MSC after 21 days of differentiation. Data shown represent means±SD, n =3 per donors.

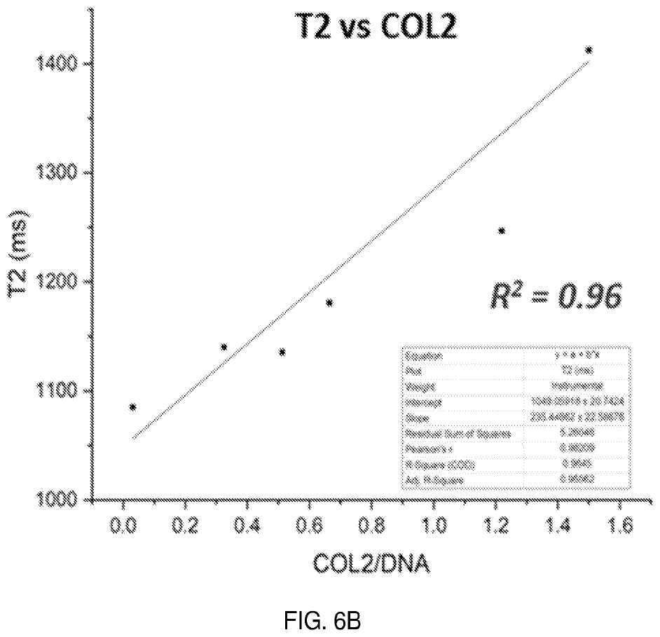

FIGS. 6A and 6B are graphs showing R² coefficient of determination of MRR T₂ levels and differentiated MSC quantitative production of sGAG or type II collagen, respectively.

The following describes the methods and the results of the disclosure.

### Materials and Methods

### MRR Measurement

MRR consists of a portable, permanent magnet (Metrolab Instruments, Plan-les-Ouates, Switzerland) with B₀=0.5 T and a bench-top type NMR console (Kea Magritek, Wellington, New Zealand). ¹H MRR measurements were performed at the resonance frequency of 21.65 MHz inside the magnet. A single resonance proton MRR probe with a detection micro-coil of 900-μm inner diameter was used for accommodating the MRR samples into the microcapillary tubes (o.d.: 1,500 μm, i.d.: 950 μm) (22-260-950, Fisherbrand, Waltham, MA, USA). In the MRR probe, the electronic parts and coil were mounted on the single printed circuit board (FIG. 1A). All the experiments were performed at 26.3° C. inside the magnet maintained by a temperature controller (RS component, UK).

For all MRR experiments, the normalized concentration (6×104 cells in 4 μL volume) of MSCs have been used unless otherwise stated. The MSC samples were spun down at 400 g for 5 minutes, and the supernatant was aspirated. The pellet is suspended in PBS with concentration of 1.5×104 cells per μL and 4 μL was filled at a 4 mm length of the micro-capillary tube. The micro- capillary tube was sealed with crystoseal (Leica Microsystems) and mounted into the coil for MRR measurements. Proton transverse relaxation times (T2) were measured by standard Carr-Purcell-Meiboom-Gill (CPMG) pulse programme (FIGS. 3A-3B). The transmitter power output was maintained at 12.5 mW for a single 90° pulse of pulse length 6 μs for all the T₂ measurements. The CPMG train of pulses with inter echo time of 200 μs with 4000 echoes was used for all experiments. A recycle delay of 2 s, which is sufficient to allow all the spins to return to thermal equilibrium, was used. Twenty-four scans were performed for all experiments for signal averaging.

### MSC Expansion

Culture expanded human bone marrow derived MSCs were purchased from Lonza Pte. Ltd. (Lonza Lot NO. 0F4452) and STEMCELL Technologies (Catalog #70022). The cells were further culture expanded in in Dulbecco's modified Eagle medium (DMEM) (Invitrogen) supplemented with 10% FBS (Invitrogen), and cultured at 37° C. in 5% CO₂ atmosphere. MSCs were harvested at 70-80% confluency by 3-min incubation with 0.25% Trypsin-EDTA (Thermo Fisher Scientific, SG) at 37° C. The cell count and viability were measured using disposable haemocytometer (INCYTO, KR) after 1:1 mixing with Trypan Blue (Thermo Fisher Scientific, SG). MSCs at passage 4 were used for MRR measurement and differentiation. Six MSC donors were used.

### MSC Chondrogenic Differentiation

Chondrogenesis of MSCs was induced in cell pellets at density of 1×10⁵ cells/pellet in chondrogenic medium, which consisted of 10⁻⁷ M dexamethasone, 50 μg/mL ascorbic acid, 1% ITS Premix supplement, 1 mM sodium pyruvate, 4 mM proline, 1% Pen-Strep, 1 mM GlutaMax and 10 ng/ml TGF-β3 in high-glucose DMEM medium (Thermo Fisher Scientific, SG). The chondrogenic medium was changed every alternative day for 3 weeks before the pellets were processed for histology analysis and matrix quantification.

### Histology Staining

The cartilage samples formed by MSCs after 21 days of chondrogenesis were fixed in 10% formalin, dehydrated, paraffin embedded, and cut into sections of 5 μm. The paraffin sections were de-waxed and rehydrated in ethanol using standard protocol. The chondrogenesis was verified by expression of (i) Proteoglycan, stained with 0.1% Safranin O Solution (Acros Organics, USA) and counterstained with 0.02% Fast green solution (Sigma-Aldrich) and Accustain® Harris hematoxylin (Sigma-Aldrich); and (ii) Type II collagen by immunohistochemical staining using mouse monoclonal antibodies (Clone 6B3 at 1:500 dilution, Chemicon, USA), followed by incubation with biotinylated goat anti-mouse (Lab Vision Corporation). Images of the staining were captured using bright field microscope (IX 71 Olympus, JP) with CCD camera (DP70, Olympus, JP).

### Cartilaginous Extracellular Matrix and DNA Quantification

Pellets harvested after 21 days of chondrogenesis were digested with 10 mg/ml of pepsin in 0.05 M acetic acid at 4° C., followed by digestion with elastase (1 mg/mL). A Blyscan sulfated glycosaminoglycan (sGAG) assay kit (Biocolor Ltd., Newtown abbey, Ireland) was used to quantify sGAG deposition according to manufacturer's protocol. Absorbance was measured at 656 nm and sGAG concentration was extrapolated from a standard curve generated using a sGAG standard. Type II Collagen (Col 2) content was measured using a captured enzyme-linked immunosorbent assay (Chondrex, Redmond, WA). Absorbance at 490 nm was measured and the concentration of Col 2 was extrapolated from a standard curve generated using a Col 2 standard. Values for sGAG and Col 2 content obtained were normalized to the total DNA content of respective samples, measured using Picogreen dsDNA assay (Molecular Probes, OR, USA). Triplicates of each MSC donor were analyzed from 7 MSC donor experiments.

### Results

### MRR Measurement of MSC Prior to Differentiation

FIG. 4 is a graph showing the MRR measurement of six MSC donors, taken just before being subjected to chondrogenic differentiation. MRR measurement indicate that there is a donor-to-donor variation of transverse relaxation time, T2, ranging from under 1100 to beyond 1400 ms.

### MSC Chondrogenic Differentiation

The extent of MSC differentiation was assessed after 21 days of chondrogenic differentiation, by both histology staining (FIG. 5A) and quantitation matrix analysis (FIG. 5B). Donor-to-donor variation in chondrogenic capacity, as indicated by different levels of cartilaginous matrix formation was observed.

### Correlation of MRR T₂ levels to chondrogenic potential of MSC

A direct correlation between MRR T₂ levels and differentiated MSC quantitative production of sGAG or type II collagen (COL2) was found (FIGS. 6A-6B, respectively), with R² coefficient of determination at 0.95 and 0.96, respectively.

The following references, many of which are cited herein, are each incorporated by reference in their entirety.

### References

Zha K, Li X, Yang Z, Tian G, Sun Z, Sui X, Dai Y, Liu S, Guo Q. Hereogeneity of mesenchymal stem cells in cartilage regeneration: from characterisation to application. NPJ Regen Med. 2021 Mar. 19;6 (1): 14.
Peng WK, Kong TF, Ng CS, Chen L, Huang Y, Bhagat AA, Nguyen NT, Preiser PR, Han J. Micromagnetic resonance relaxometry for rapid label-free malaria diagnosis. Nat Med. 2014 September;20 (9): 1069-73.
Jones EA, Kinsey SE, English A, Jones RA, Straszynski L, Meredith DM, Markham AF, Jack A, Emery P, McGonagle D. Isolation and characterization of bone marrow multipotential mesenchymal progenitor cells. Arthritis Rheum. 2002 December; 46 (12): 3349-60.
Chang CB, Han SA, Kim EM, Lee S, Seong SC, Lee MC. Chondrogenic potentials of human synovium-derived cells sorted by specific surface markers. Osteoarthritis Cartilage. 2013 January;21 (1): 190-9.
Mifune Y, Matsumoto T, Murasawa S, Kawamoto A, Kuroda R, Shoji T, Kuroda T, Fukui T, Kawakami Y, Kurosaka M, Asahara T. Therapeutic superiority for cartilage repair by CD271-positive marrow stromal cell transplantation. Cell Transplant. 2013;22 (7): 1201-11.
Mabuchi Y, Morikawa S, Harada S, Niibe K, Suzuki S, Renault-Mihara F, Houlihan DD, Akazawa C, Okano H, Matsuzaki Y. LNGFR(+)THY-1(+)VCAM-1(hi+) cells reveal functionally distinct subpopulations in mesenchymal stem cells Stem Cell Reports. 2013 Jul. 11;1 (2): 152-65.
Li X, Guo W, Zha K, Jing X, Wang M, Zhang Y, Hao C, Gao S, Chen M, Yuan Z, Wang Z, Zhang X, Shen S, Li H, Zhang B, Xian H, Zhang Y, Sui X, Qin L, Peng J, Liu S, Lu S, Guo Q. Enrichment of CD146(+) Adipose-Derived Stem Cells in Combination with Articular Cartilage Extracellular Matrix Scaffold Promotes Cartilage Regeneration. Theranostics. 2019 Jul. 9;9 (17): 5105-5121.
Sathiyanathan P, Samsonraj RM, Tan CLL, Ling L, Lezhava A, Nurcombe V, Stanton LW, Cool SM. A genomic biomarker that identifies human bone marrow-derived mesenchymal stem cells with high scalability. Stem Cells. 2020 September;38 (9): 1124-1136.
Peng, W. K. et al. Micromagnetic resonance relaxometry for rapid label-free malaria diagnosis. Nat. Med. 20, 1069-1073, doi: 10.1038/nm.3622 (2014).
Carr, H. Y. & Purcell, E. M. Effects of diffusion on free precession in nuclear magnetic resonance experiments. Physical review 94, 630 (1954).
Meiboom, S. & Gill, D. Modified spin-echo method for measuring nuclear relaxation times. Rev. Sci. Instrum. 29, 688-691 (1958).

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A method of identifying chondrogenic potential of mesenchymal stem cells comprising:
loading a liquid sample including a plurality of mesenchymal stem cells in a sensor;
placing the sensor including the liquid sample within or nearby a detection coil of a magnetic resonance relaxometry device;
determining a T2 value for the liquid sample; and
identifying mesenchymal stem cells with high chondrogenic potential when the T2 value is greater than or equal to a first threshold value and mesenchymal stem cells with low chondrogenic potential when the T2 value is less than or equal to a second threshold value.

2. The method according to claim 1, wherein the sensor is comprises a tube or chamber.

3. The method according to claim 1, wherein the first threshold value is greater than the second threshold value.

4. The method according to claim 1, wherein a gap between the first threshold value and the second threshold value is 10 ms, 20 ms, 30 ms, 40 ms, 50 ms, 60 ms, 70 ms, 80 ms, 90 ms or 100 ms.

5. The method according to claim 1, wherein the first threshold value is 1200 ms.

6. The method according to claim 1, wherein the second threshold value is 1100 ms.

7. The method according to claim 1, wherein the T2 value is correlated with the formation of a cartilaginous matrix component.

8. The method according to claim 7, wherein the cartilaginous matrix component includes sulphated glycoaminoglycan (sGAG), type II collagen (COL2), or mixtures thereof.

9. The method according to claim 1, wherein the T2 value is correlated with quantitative production of sGAG.

10. The method according to claim 1, wherein the T2 value is correlated with quantitative production of COL2.

11. The method according to claim 1, wherein the mesenchymal stem cells include bone marrow derived mesenchymal stem cells.

12. (canceled)

13. The method according to claim 1, wherein determining the T2 value includes obtaining and averaging 2 to 70 scans.

14. The method according to claim 1, further comprising separating mesenchymal stem cells with high chondrogenic potential from mesenchymal stem cells with low chondrogenic potential.

15. A system for identifying chondrogenic potential of mesenchymal stem cells comprising:
a magnetic resonance relaxometry device configured to determine a T2 value for a liquid sample including a plurality of mesenchymal stem cells in a sensor and to identify mesenchymal stem cells with high chondrogenic potential when the T2 value is greater than or equal to a first threshold value and mesenchymal stem cells with low chondrogenic potential when the T2 value is less than or equal to a second threshold value.

16. The system of claim 15, further comprising a cell production device as a source for the plurality of mesenchymal stem cells.

17. The system of claim 16, wherein the cell production device includes a cell culture vessel.

18. The system of claim 17, wherein the cell culture vessel includes a plate, a well, a chamber, or a combination thereof.

19. The system of claim 16, wherein the cell production device includes an incubator.

20. The system of claim 15, further comprising a cell separation device.

21. The system of claim 20, wherein the cell separation device includes a microfluidic device and/or a cell sorter.

22. (canceled)
